(19) United States
(12) Patent Application Publication
(10) Pub. No.: US 2008/0009429 A1
(43) Pub. Date: Jan. 10, 2008

(54) Cosmetic or dermatological cleansing compositions comprising secondary alkanesulfonates

(75) Inventors: Peter Klug, Grossostheim (DE); Waltraud Simsch, Kelkheim (DE); Vera Mulitze-Kleinheyer, Frankfurt am Main (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 11/824,820

(22) Filed: Jul. 3, 2007

(30) Foreign Application Priority Data
Jul. 6, 2006 (DE) 102006031377.1

(51) Int. Cl. A61K 8/00

(52) U.S. Cl. 510/130

(57) ABSTRACT

Stable liquid cosmetic or dermatological cleansing compositions comprising one or more secondary alkanesulfonates and one or more betaines are described, wherein the weight ratio of alkanesulfonate to betaine is from 6:3 to 1:2 and the viscosity of the cleansing composition is between 1000 and 30 000 mPas.

Cosmetic or dermatological cleansing compositions comprising secondary alkanesulfonates

The present invention relates to cosmetic, pharmaceutical and dermatological compositions comprising secondary alkanesulfonates and betaines.

The wishes of consumers, dermatological and toxicological aspects, and the rheology of cosmetic products are closely linked to one another. Thus skin friendly products, harmless from the toxicological and ecotoxicological point of view, having favorable Theological properties are demanded.

Secondary alkanesulfonates have been known for a long time as basic surfactants, especially for detergent applications and industrial cleansers.

In US 2004/0 204 336, aqueous liquid dispersions are disclosed, comprising secondary alkanesulfonates, builders and electrolytes, stabilized by an alkylhydroxyethylammonium salt.

In US 2004/0 029 757, manual dishwashing liquid formulations are described, the surfactant mixture consisting of secondary alkanesulfonates, alkyl ether sulfates, alkyl sulfates, alkylmethyl ester sulfates, α-olefinsulfonates and a betaine.

DE 101 62 648 discloses sprayable liquid aqueous cleansers, comprising a surfactant combination of alkyl ether sulfate, secondary alkanesulfonate and amphosurfactant.

In WO 2006/050 875, hair treatment compositions are described, which contain alkyl ether sulfate or alkyl sulfate or a mixture thereof, secondary alkanesulfonate, betaine or ethercarboxylate or a mixture thereof, one or more nonionic surfactants and a cationic polymer.

In cosmetic and pharmaceutical products, secondary alkanesulfonate was employed in the past.

In contrast to alkyl ether sulfates, for example sodium lauryl ether sulfate, or alkyl sulfates, for example sodium lauryl sulfate, secondary alkanesulfonates have the disadvantage that they do not react to the customary methods of thickening, especially to the addition of common salt, with a viscosity increase, even in the presence of cosurfactants such as, for example, betaines. This applies especially in the customary ratios of basic surfactants to betaine of 7 to 3 or 8 to 2 and total surfactant contents of below 20% by weight in the formulation.

The object was to make available surfactant systems for liquid cosmetic formulations, in particular for the cleansing of the skin and hair, which are adjustable to viscosities in the range from 1000 to 30 000 mPas even at surfactant concentrations of below 20% by weight and at the same time are skin friendly and toxicologically harmless.

Surprisingly, it has been found that on combination of secondary alkanesulfonates with betaines very definitely viscous surfactant systems can be obtained if the weight ratio of secondary alkanesulfonate to betaine is chosen in the range from 6:3 to 1:2. These systems are very readily handleable, in particular at pHs of below 6, and form colorless, clear, and stable surfactant solutions, which can be used as cosmetic and pharmaceutical products in the form of shampoos, shower gels or liquid soaps.

The invention relates to stable liquid cosmetic or dermatological cleansing compositions comprising one or more secondary alkanesulfonates and one or more betaines, wherein the weight ratio of alkanesulfonate to betaine is from 6:3 to 1:2 and the viscosity of the cleansing composition is between 1000 and 30 000 mPas.

Stable cleansing compositions in the context of the present invention are formulations which show no separation, precipitation and turbidity at temperatures in the range from 0 to +50° C. and even with storage times of over several months.

The viscosities were determined by means of a Brookfield viscometer model DV-11 with spindle no. 3 at a speed of rotation of 20 1/s.

The cleansing composition according to the invention is distinguished by a good cleansing power, a good skin mildness and an esthetic appearance.

The weight ratio alkanesulfonate to betaine in the cleansing compositions according to the invention is preferably from 4:3 to 1:1.

The viscosity in the cleansing compositions according to the invention is preferably between 2000 and 10 000 mPas.

The amount of the one or more secondary alkanesulfonates and the one or more betaines in the cleansing compositions according to the invention together, based on the total weight of the compositions, is preferably between 6 and 20% by weight and particularly preferably between 8 and 15% by weight.

In a preferred embodiment of the invention, the one or more betaines is/are selected from alkylamidopropylbetaines according to formula (1)

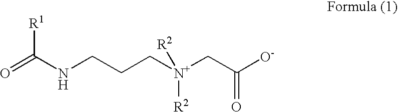

and alkylbetaines according to formula (2)

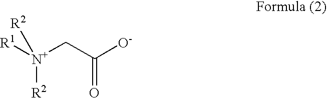

in which R1 is an alkyl, hydroxyalkyl or alkylphenyl group, preferably an alkyl group, having 8 to 22 carbon atoms and each radical R2 is a methyl group.

In a further preferred embodiment of the invention, the one or more betaines is/are selected from the group consisting of alkylamidopropylbetaines having 8 to 18 carbon atoms in the alkyl group of the fatty acid radical, alkylbetaines having 8 to 18 carbons in the alkyl group and mixtures of these substances.

In a particularly preferred embodiment of the invention, the one or more betaines is/are selected from alkylamidopropylbetaine or a mixture of alkylamidopropylbetaine and alkylbetaine.

In a further preferred embodiment of the invention, the cleansing compositions are clear.

In the cleansing compositions according to the invention, secondary alkanesulfonates are employed whose alkyl group is saturated or unsaturated, linear or branched and which can optionally also carry hydroxyl groups, the terminal carbon atoms of the alkyl chain having no sulfonate group.

Secondary alkanesulfonates with linear alkyl groups having 8 to 20 carbon atoms are preferred, which carry one or more SO3X groups randomly distributed in the secondary position on the hydrocarbon chain. The counterion X can be a sodium, potassium, ammonium, mono-, di- or triammonium, calcium or magnesium ion or a mixture of the counterions mentioned. Sodium salts of the secondary alkanesulfonates are preferred.

In a further preferred embodiment of the invention, the one or more secondary alkanesulfonates is/are selected from C8-20 alkanesulfonates.

In a particularly preferred embodiment of the invention, the one or more secondary alkanesulfonates is/are selected from C14-17 alkanesulfonates, preferably from their sodium salts.

Alkoxylated surfactants, in particular alkyl ether sulfates, due to preparation, contain short-chain glycols in fairly small amounts, which are assessed as toxicologically harmful, so that in the market a demand for “EO-free”, i.e. ethylene oxide-free, or “ether sulfate-free” formulations exists.

The cleansing compositions according to the invention are distinguished in that they must contain no ethoxylated or propoxylated compounds. In a further preferred embodiment of the invention, the cleansing compositions therefore contain no ethoxylated or propoxylated compounds. In a particularly preferred embodiment of the invention, the cleansing compositions contain no alkyl ether sulfates.

In a further preferred embodiment of the invention, the cleansing compositions contain, in addition to the one or more secondary alkanesulfonates and the one or more betaines, one or more cosurfactants, preferably selected from the group of anionic, cationic, nonionic, zwitterionic and amphotenc surfactants.

Suitable additional anionic surfactants are preferably (C10-C20)alkyl- and alkylene-carboxylates, alkyl ether carboxylates, fatty alcohol sulfates, fatty alcohol ether sulfates, alkylamide sulfates and sulfonates, fatty acid alkylamide polyglycol ether sulfates, olefin sulfonates, acyl esters of isethionates, α-sulfofatty acid esters, alkylbenzenesulfonates, alkylphenol glycol ether sulfonates, sulfosuccinates, sulfo-succinic acid hemiesters and diesters, fatty alcohol ether phosphates, protein-fatty acid condensation products, alkylmonoglyceride sulfates and sulfonates, alkylglyceride ether sulfonates, fatty acid methyltaurides, fatty acid sarcosinates, sulforicinoleates, acylglutamates.

The compounds and their mixtures are used in the form of their water-soluble or water-dispersible salts, for example of the sodium, potassium, magnesium, ammonium, mono-, di- and triethanolammonium, and of the analogous alkylammonium salts.

The weight proportion of the additional anionic surfactants, based on the total weight of the composition, is preferably from 0.5 to 10% by weight, particularly preferably from 1 to 8% by weight and especially preferably from 2 to 5% by weight.

Suitable cationic surfactants are, for example, quaternary ammonium salts such as di(C10-C24)alkyldimethylammonium chloride or bromide, preferably di(C12-C18)alkyldi-methylammonium chloride or bromide; (C10-C24)alkyldimethylethylammonium chloride or bromide; (C10-C24)alkyltrimethylammonium chloride or bromide, preferably cetyltrimethylammonium chloride or bromide and (C20-C22)alkyl-trimethylammonium chloride or bromide; (C10-C24)alkyldimethylbenzylammonium chloride or bromide, preferably (C12-C18)alkyldimethylbenzylammonium chloride; N-(C10-C18)alkylpyridinium chloride or bromide, preferably N-(C12-C16)alkylpyridinium chloride or bromide; N-(C10-C18)alkylisoquinolinium chloride, bromide or monoalkyl-sulfate; N-(C12-C18)alkylpolyoylaminoformylmethylpyridinium chloride; N-(C12-C18)alkyl-N-methylmorpholinium chloride, bromide or monoalkylsulfate; N-(C12-C18)alkyl-N-ethylmorpholinium chloride, bromide or monoalkylsulfate; (C16-C18)alkylpenta-oxethylammonium chloride; diisobutylphenoxyethoxyethyidimethylbenzylammonium chloride; salts of N,N-diethylaminoethylstearylamide and oleylamide with hydrochloric acid, acetic acid, lactic acid, citric acid, phosphoric acid; N-acylaminoethyl-N,N-diethyl-N-methylammonium chloride, bromide or monoalkylsulfate and N-acylamino-ethyl-N,N-diethyl-N-benzylammonium chloride, bromide or monoalkylsulfate, acyl preferably being stearyl or oleyl.

The weight proportion of the cationic surfactants, based on the total weight of the compositions, is preferably from 0.5 to 10% by weight and particularly preferably from 1 to 5% by weight.

Suitable nonionic surfactants are preferably N-alkyl, N-alkoxypolyhydroxyfatty acid amide, fatty acid N-alkylglucamides, alkylpolyglucoside, alkylphenol polyethylene glycols; alkylmercaptan polyethylene glycols, fatty alcohol ethoxylates, fatty amine ethoxylates (alkylaminopolyethylene glycols), polypropylene glycol ethoxylates (Pluronics®), sucrose esters; sorbitol esters and polyglycol ethers.

The weight proportion of the nonionic surfactants, based on the total weight of the compositions, is preferably from 0.5 to 10% by weight, particularly preferably from 1 to 5% by weight and especially preferably from 2 to 4% by weight.

Preferred amphosurfactants are acylglutamates, amphoacetates, N-(C12-C18)alkyl-β-aminopropionates and N-(C12-C18)alkyl-β-iminodipropionates as the alkali metal and mono-, di- and trialkylammonium salts; amphosurfactants based on imidazoline (trade name: Miranol®, Steinapon®), preferably the sodium salt of 1-(β-carboxy-methyloxyethyl)-1-(carboxymethyl)-2-laurylimidazolinium; amine oxide, e.g. (C12-C18)-alkyldimethylamine oxide, fatty acid amidoalkyldimethylamine oxide.

The weight proportion of the additional amphoteric surfactants, based on the total weight of the compositions, is preferably from 0.5 to 10% by weight and particularly preferably from 1 to 8% by weight.

Particularly preferred cleansing compositions according to the invention contain one or more cosurfactants, which are selected from acylglutamates, alkylpolyglucosides, amphoacetates, coconut monoethanolamides, coconut isopropanolamides or mixtures of these substances.

The total amount of the surfactants employed, that is the sum of secondary alkanesulfonatess, betaines and cosurfactants, is, if the latter are contained in the composition and based on the total weight of the composition, preferably from 6.5 to 20% by weight, particularly preferably from 8 to 18% by weight and especially preferably from 10 to 16% by weight.

The compositions according to the invention can contain, as further excipients and additives, oily substances, emulsifiers and coemulsifiers, and further additives customary in the cosmetic, pharmaceutical and dermatological field, such as, for example, cationic polymers, film-forming agents, superfatting agents, stabilizers, biogenic active compounds, glycerol, preservatives, pearl luster agents, colorants and scents, solvents, opacifiers, further protein derivatives such as gelatin, collagen hydrolyzates, naturally and synthetically based polypeptides, egg yolk, lecithin, lanolin and lanolin derivatives, fatty alcohols, silicones, deodorants, substances having keratolytic and keratoplastic action, enzymes and vehicles. In addition, antimicrobial agents can be added to the compositions according to the invention.

Oily substance is to be understood as meaning any fatty substance which is liquid at room temperature (25° C.).

The fatty phase can therefore comprise one or more oils, which are preferably selected from the following oils:

silicone oils, volatile or nonvolatile, linear, branched or cyclic, possibly organically modified; phenylsilicones; silicone resins and gums; mineral oils such as paraffin or petroleum oil; oils of animal origin such as perhydrosqualene, lanolin;

oils of vegetable origin such as liquid triglycerides, e.g. sunflower, corn, soybean, rice, jojoba, babusscu, pumpkin, grapeseed, sesame, walnut, apricot, macadamia, avocado, sweet almond, lady's smock and castor oil, triglycerides of caprylic/capric acids, olive oil, peanut oil, rapeseed oil and coconut oil;

synthetic oils such as purcellin oil, isoparaffins, linear and/or branched fatty alcohols and fatty acid esters, preferably Guerbet alcohols having 6 to 18, preferably 8 to 10, carbon atoms; esters of linear (C6-C13)-fatty acids with linear (C6-C20)-fatty alcohols; esters of branched (C6-C13)-carboxylic acids with linear (C6-C20)-fatty alcohols, esters of linear (C6-C18)-fatty acids with branched alcohols, in particular 2-ethylhexanol; esters of linear and/or branched fatty acids with polyhydric alcohols (such as, for example, dimer diol or trimer diol) and/or Guerbet alcohols; triglycerides based on (C6-C10)-fatty acids;

esters such as dioctyl adipate, diisopropyl dimer dilinoleate; propylene glycols/dicaprylate or waxes such as beeswax, paraffin wax or microwaxes, optionally in combination mit hydrophilic waxes, such as for example cetylstearyl alcohol; fluorinated and perfluorinated oils; fluorinated silicone oils; mixtures of the aforementioned compounds.

Suitable cationic polymers are the substances known under the INCl name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and Polyquaternium 37 & mineral oil & PPG trideceth (Salcare SC95), PVP-dimethylaminoethyl methacrylate copolymer, guar hydroxypropyltriammonium chlorides, and calcium alginate and ammonium alginate. In addition, cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethylenimines; cationic silicone polymers, such as, for example, amidomethicone; copolymers of adipic acid and dimethylaminohydroxy-propyldiethylenetriamine; polyaminopolyamide and cationic chitin derivatives, such as, for example, chitosan can be employed.

Suitable silicone compounds are, for example, dimethylpolysiloxane, methylphenylpolysiloxanes, cyclic silicones and amino-, fatty acid-, alcohol-, polyether-, epoxy-, fluorine- and/or alkyl-modified silicone compounds, and also polyalkylsiloxanes, polyalkylarylsiloxanes, polyethersiloxane copolymers, such as described in U.S. Pat. No. 5,104,645 and the specifications cited therein, which can be present both in liquid and resinous form at room temperature.

Suitable film-forming agents, depending on the intended use, are salts of phenylbenzimidazolesulfonic acid, water-soluble polyurethanes, for example C10-polycarbamyl polyglycerol esters, polyvinyl alcohol, polyvinylpyrrolidone, copolymers, for example vinylpyrrolidone/vinyl acetate copolymer, water-soluble acrylic acid polymers/copolymers and their esters or salts, for example partial ester copolymers of acrylic/methacrylic acid and polyethylene glycol ethers of fatty alcohols, such as acrylate/steareth-20 methacrylate copolymer, water-soluble cellulose, for example hydroxymethylcellulose, hydroxyethylcellulose, hydroxypropylcellulose, water-soluble quaterniums, polyquaterniums, carboxyvinyl polymers, such as carbomers and their salts, polysaccharides, for example polydextrose and glucan.

Superfatting agents which can be used are substances such as, for example, polyethoxylated lanolin derivatives, lecithin derivatives, polyol fatty acid esters, monoglycerides and fatty acid alkanolamides, the latter simultaneously serving as foam stabilizers. An available moisturizing substance is, for example, isopropyl palmitate, glycerol and/or sorbitol.

Stabilizers which can be employed are metal salts of fatty acids, such as, for example, magnesium, aluminum and/or zinc stearate.

Biogenic active compounds are to be understood as meaning, for example, plant extracts and vitamin complexes.

The compositions according to the invention can additionally contain organic solvents. In principle, suitable organic solvents are all mono- or polyhydric alcohols. Preferably, alcohols having 1 to 4 carbon atoms such as ethanol, propanol, isopropanol, n-butanol, i-butanol, t-butanol, glycerol and mixtures of the alcohols mentioned are employed. Further preferred alcohols are polyethylene glycols having a relative molecular mass below 2000. In particular, use of polyethylene glycol having a relative molecular mass between 200 and 600 and in amounts up to 45% by weight and of polyethylene glycol having a relative molecular mass between 400 and 600 in amounts of 5 to 25% by weight is preferred. Further suitable solvents are, for example, triacetin (glycerol triacetate) and 1-methoxy-2-propanol. Short-chain anionic surfactants, in particular arylsulfonates, for example cumene- or toluenesulfonate, have a hydrotropic action.

The compositions according to the invention can be blended with conventional ceramides, pseudoceramides, fatty acid N-alkylpolyhydroxyalkylamides, cholesterol, cholesterol fatty acid esters, fatty acids, triglycerides, cerebrosides, phospholipids and similar substances as a care additive.

Suitable preservatives are, for example, phenoxyethanol, parabens, pentanediol, octanediol, benzoic acid, salicylic acid or sorbic acid.

Colorants which can be used are the substances suitable and permitted for cosmetic purposes.

Suitable fungicidal active compounds are preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione and octopirox (Clariant).

In a further preferred embodiment of the invention, the cleansing compositions contain one or more UV filters.

Suitable UV filters are preferably 4-aminobenzoic acid; 3-(4′-trimethylammonium)-benzylideneboran-2-one methylsulfate; 3,3,5-trimethylcyclohexyl salicylate; 2-hydroxy-4-methoxybenzophenone; 2-phenylbenzimidazole-5-sulfonic acid and its potassium, sodium and triethanolamine salts; 3,3′-(1,4-phenylenedimethine)bis(7,7-dimethyl-2-oxobicyclo[2.2.1]heptane-1-methanesulfonic acid and its salts; 1-(4-tert-butylphenyl)-3-(4-methoxyphenyl)propane-1,3-dione, 3-(4′-sulfo)benzylidenebornan-2-one and its salts; 2-cyano-3,3-diphenylacrylic acid 2-ethylhexyl ester; polymer of N-[2(and 4)-(2-oxoborn-3-ylidenemethyl)benzyl]acrylamide; 4-methoxycinnamic acid 2-ethylhexyl ester; ethoxylated ethyl 4-aminobenzoate; 4-methoxycinnamic acid isoamyl ester; 2,4,6-tris[p-(2-ethylhexyloxycarbonyl)anilino]-1,3,5-triazine; 2-(2N-benzotriazol-2-yl)-4-methyl-6-(2-methyl-3-(1,3,3,3-tetramethyl-1-(trimethylsilyloxy)-disiloxanyl)propyl)phenol; 4,4′-[(6-[4-((1,1-dimethylethyl)aminocarbonyl)phenyl-amino]-1,3,5-triazin-2,4-yl)diimino]bis(benzoic acid 2-ethylhexyl ester); 3-(4′-methyl-benzylidene)-D,L-camphor; 3-benzylidenecamphor; salicylic acid 2-ethylhexyl ester; 4-dimethylaminobenzoic acid 2-ethylhexyl ester; hydroxy-4-methoxybenzophenone-5-sulfonic acid (sulisobenzonum) and the sodium salt; and/or 4-isopropylbenzyl salicylate, N,N,N-trimethyl-4-(2-oxoborn-3-ylidenemethyl)anilinium methylsulfate, homosalate (INN) oxybenzone (INN), 2-phenylbenzimidazole-5-sulfonic acid and its Na, K and triethanolamine salts, alpha-(2-oxoborn-3-ylidene)toluene-4-sulfonic acid and its salts, octylmethoxycinnamic acid, isopentyl-4-methoxycinnamic acid, isoamyl-p-methoxycinnamic acid, 2,4,6-trianilino-(p-carbo-2′-ethylhexyl-1′-oxy)-1,3,5-triazine (octyltriazone)phenol, 2-2(2H-benzotriazol-2-yl)-4-methyl-6-(2-methyl-3-(1,3,3,3-tetramethyl-1-(trimethylsilyl)oxy)disiloxanyl)propyl (drometriazole trisiloxane) benzoic acid, 4,4-((6-(((1,1-dimethylethyl)amino)carbonyl)phenyl)amino)-1,3,5-triazine-2,4-diyl)diimino)bis, bis(2-ethylhexyl)ester) benzoic acid, 4,4-((6-(((1,1-dimethylethyl)-amino)carbonyl)phenyl)amino)-1,3,5-triazine-2,4-diyl)diimino)bis, bis(2-ethylhexyl)ester) 3-(4′-methylbenzylidene)-d,l-camphor (4-methylbenzylidene camphor), 3-benzylidene camphor (3-benzylidene camphor), 2-ethylhexyl salicylate (octyl salicylate), ethyl-2-hexyl 4-dimethylaminobenzoate (octyldimethyl PABA), 2-hydroxy-4-methoxybenzophenone-5-sulfonic acid (benzophenone-5) and the Na salt, 2,2′-methylenebis-6-(2H-benzotriazol-2-yl)-4-(tetramethylbutyl)-1,1,3,3-phenol, sodium salt of 2-2′-bis(1,4-phenylene)1H-benzimidazole-4,6-disulfonic acid, (1,3,5)-triazine-2,4-bis((4-(2-ethylhexyloxy)-2-hydroxy)phenyl)-6-(4-methoxyphenyl), 2-ethylhexyl 2-cyano-3,3-diphenyl-2-propenoate, glyceryl octanoate di-p-methoxycinnamic acid, p-aminobenzoic acid and ester, 4-tert-butyl-4′-methoxydibenzoylmethane, 4-(2-[beta]-glucopyranoxy)propoxy-2-hydroxybenzophenone, octyl salicylate, methyl-2,5-diisopropylcinnamic acid, cinoxate, dihydroxydimethoxybenzophenone, disodium salt of 2,2′-dihydroxy-4,4′-dimethoxy-5,5′-disulfobenzophenone, dihydroxybenzophenone, 1-3,4-dimethoxyphenyl)-4,4-dimethyl-1,3-pentanedione, 2-ethylhexyl dimethoxy-benzylidene dioxoimidazolidine propionate, tetrahydroxybenzophenone, terephthalidenedicamphorsulfonic acid, 2,4,6-tris[4-2-ethylhexyloxycarbonyl)anilino]-1,3,5-triazine, methylbis(trimethylsiloxy)silylisopentyltrimethoxycinnamic acid, amyl p-dimethylaminobenzoate, amyl p-dimethylaminobenzoate, 2-ethylhexyl p-dimethyl-aminobenzoate, isopropyl p-methoxycinnamic acid/diisopropylcinnamic acid ester, 2-ethylhexyl p-methoxycinnamic acid, 2-hydroxy-4-methoxybenzophenone, 2-hydroxy-4-methoxybenzophenone-5-sulfonic acid and the trihydrate, 2-hydroxy-4-methoxybenzophenone-5-sulfonate, Na salt, phenylbenzimidazolesulfonic acid.

The compositions according to the invention contain UV lightscreen filters in the amounts of preferably 0.1 to 10% by weight, particularly preferably 0.5 to 8% by weight and especially preferably 1 to 5% by weight, based on the total weight of the compositions.

In a further preferred embodiment of the invention, the cleansing compositions contain one or more antioxidants.

Advantageously, the antioxidants are chosen from the group consisting of amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotenoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and its derivatives (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerable doses, furthermore (metal) chelators (e.g. a-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutic acid and its derivatives, α-glycosylrutin, ferulic acid, furfurylideneglucitol, carnosine, butyl-hydroxytoluene, butylhydroxyanisole, nordihydroguaiaretic acid, trihydroxy-butyrophenone, uric acid and its derivatives, mannose and its derivatives, zinc and its derivatives (e.g. ZnO, ZnSO4), selenium and its derivatives (e.g. selenomethionine), stilbenes and their derivatives (e.g. stilbene oxide, trans-stilbene oxide), superoxide dismutase and the derivatives suitable according to the invention (salts, esters, ethers, sugars, nucleotides, nucleosides, peptides and lipids) of said substances.

Water-soluble antioxidants can be employed particularly advantageously within the meaning of the present invention.

The antioxidants can protect the skin and the hair from oxidative stress. Preferred antioxidants here are vitamin E and its derivatives and vitamin A and its derivatives.

The amount of the one or more antioxidants in the compositions according to the invention is preferably from 0.001 to 30% by weight, particularly preferably from 0.05 to 20% by weight and especially preferably from 1 to 10% by weight, based on the total weight of the compositions.

If vitamin E and/or its derivatives are the antioxidant(s), it is advantageous to choose their respective concentrations from the range from 0.001 to 10% by weight, based on the total weight of the compositions.

If vitamin A, or vitamin A derivatives, or carotenes or their derivatives are the antioxidant(s), it is advantageous to choose their respective concentrations from the range from 0.001 to 10% by weight, based on the total weight of the compositions.

In a particularly preferred embodiment of the invention, the compositions according to the invention contain antioxidants selected from superoxide dismutase, tocopherol (vitamin E) and ascorbic acid (vitamin C).

The compositions according to the invention are preferably rinse-off formulations, in particular shampoos, shower baths, shower gels, foam baths and liquid soaps. Modern rinse-off products often have a high content of conditioning active compounds, which can consist of oil fractions. Consequently, these compositions can be present as emulsions.

In a particularly preferred embodiment of the invention, the cleansing compositions are shampoos, shower baths or liquid soaps.

The following examples are intended to illustrate the invention in more detail, without restricting it thereto (the percentages are % by weight). Viscosities were measured by means of a Brookfield viscometer (spindle no. 3).

### EXAMPLES

### Example 1

Hair shampoo, clear, 11.15% active surfactant content

| | | | |
|---|---|---|---|
| A | Hostapur ® SAS 60 | (Clariant) | 10.00% |
| | secondary C14-17 alkylsulfonate, Na salt | | |
| | Lamesoft ® PO 65 | | 1.00% |
| | coconut glucoside and glyceryl oleate | | |
| | scent | | 0.30% |
| B | water | | to 100.00% |
| C | Genagen ® CAB | (Clariant) | 15.00% |
| | cocamidopropyl betaine | | |
| | preservative | | q.s. |
| D | citric acid | | q.s. |

### Preparation:

I mixing of the components A

II addition of B to I with stirring until a clear solution is obtained

III addition of C to II with stirring

IV adjustment of the pH to 5.5-6.0 using D

Viscosity Brookfield pH 5.7: 3600 mPas

### Comparative Example 1

Ingredients and manner of preparation analogous to Example 1. The weight ratio of secondary alkanesulfonate to alkylamidopropylbetaine with identical total active content was changed from 6:4.5=1.33 to 7:3=2.33. The formulation had a viscosity of only 70 mPas at pH=5.7.

### Example 2

Hair shampoo for men, clear, 15.5% active surfactant content

| | | | |
|---|---|---|---|
| A | Hostapur ® SAS 60 | (Clariant) | 8.00% |
| | secondary C14-17 alkylsulfonate, Na salt | | |
| | Hostapon ® CGN | (Clariant) | 10.00% |
| | cocoyl glutamate, Na salt | | |
| | Plantacare 818 | | 3.00% |
| | coconut glucoside | | |
| | Rewomid IPP 240 | | 2.00% |
| | Cocamide MIPA | | |
| | water | | 20.00% |
| B | scent | | 0.30% |
| C | water | | to 100.00% |
| D | Genagen ® CAB | (Clariant) | 8.00% |
| | cocamidopropyl betaine | | |
| | Genagen ® KB | (Clariant) | 6.00% |
| | coconut betaine | | |
| | preservative | | q.s. |
| E | citric acid | | q.s. |

Preparation:

I dissolution of A with stirring at about 50° C.

II addition of B to I with stirring at about 35° C.

III addition of C to II with stirring until a clear solution is obtained

IV addition of the components D to III with stirring

V adjustment to pH 5.7-6.3 using E

Viscosity Brookfield pH 6.3: 4430 mPas

### Example 3

Mild hair shampoo, clear, 15.5% active surfactant content

| | | | |
|---|---|---|---|
| A | Hostapur ® SAS 60 | (Clariant) | 8.00% |
| | secondary C14-17 alkylsulfonate, Na salt | | |
| | Hostapon ® CGN | (Clariant) | 10.00% |
| | cocoyl glutamate, Na salt | | |
| | Rewomid IPP 240 | | 2.00% |
| | Cocamid MIPA | | |
| | Plantacare 818 | | 3.00% |
| | coconut glucoside | | |
| | water | | 20.00% |
| B | scent | | 0.30% |
| C | water | | to 100.00% |
| | glycerol | | 1.00% |
| | sorbitol | | 0.50% |
| | panthenol | | 1.00% |
| D | Genagen ® CAB | (Clariant) | 8.00% |
| | cocamidopropyl betaine | | |
| | Genagen ® KB | (Clariant) | 6.00% |
| | coconut betaine | | |
| E | Mackpro SLP | | 0.50% |
| | Quaternium 79 hydrolyzed soybean | | |
| | protein | | |
| | preservative | | q.s. |
| F | citric acid | | q.s. |

Preparation:
I dissolution of A with stirring at about 50° C.
II addition of B with stirring at about 35° C.
III addition of the components C to II with stirring until a clear solution is formed
IV addition of the components D to III
V addition of E to IV

VI adjustment of the pH to 5.5-6.0 using F

Viscosity Brookfield: pH 6.0 3720 mPas

### Example 4

Shower bath, clear, 16.75% active surfactant content

| | | | |
|---|---|---|---|
| A | Hostapur ® SAS 60 | (Clariant) | 10.00% |
| | secondary C14-17 alkylsulfonate, Na salt | | |
| | Hostapon ® CGN | (Clariant) | 10.00% |
| | cocoyl glutamate, Na salt | | |
| | Lamesoft ® PO 65 | | 1.00% |
| | coconut glucoside and glyceryl oleate | | |
| | Rewomid IPP 240 | | 1.00% |
| | Cocamide MIPA | | |
| | Plantacare 818 | | 2.00% |
| | coconut glucoside | | |
| | water | | 20.00% |
| B | scent | | 0.50% |
| C | water | | to 100.00% |
| | glycerol | | 1.00% |
| | sorbitol | | 1.00% |
| | avocado special | | 1.00% |
| | water, ethoxydiglycol, propylene | | |
| | glycol, butylene glycol, | | |
| | Persea gratissima extract | | |
| D | Genagen ® CAB | (Clariant) | 10.00% |
| | cocamidopropyl betaine | | |
| | Genagen ® KB | (Clariant) | 7.00% |
| | coconut betaine | | |
| | preservative | | q.s. |
| E | citric acid | | q.s. |

Preparation:
I dissolution of A with stirring at about 50° C.
II stirring in of B at about 35° C.
III addition of the components C to II and stirring until a clear solution is formed
IV addition of the components D to III
adjustment of the pH to 5.5-6.0 using E

Viscosity Brookfield: pH 6.0 4120 mPas

INCl list and concentrations of the ingredients. The concentration data in this application are in each case based on the active content of the surfactants.

| | |
|---|---|
| Genagen ® CAB | cocamidopropylbetaine, 30% solution |
| Genagen ® KB | coconut betaine, 30% solution |
| Hostapur ® SAS 60 | secondary C14-17 alkylsulfonate, 60% paste |
| Plantacare 818 | coconut glucoside, 50% aq. solution |
| Rewomid IPP 240 | cocamide MIPA, 100% active |
| Lamesoft ® PO 65 | coconut glucoside and glyceryl oleate, |
| | 65% solution |
| Hostapon ® CGN | sodium cocoyl glutamate, 30% solution |
| Mackpro SLP | Quaternium 79 |

1. A stable liquid cosmetic or dermatological cleansing composition comprising one or more secondary alkanesulfonates and one or more betaines, wherein the stable liquid composition has a weight ratio of alkanesulfonate to betaine of from 6:3 to 1:2 and a viscosity of is between 1000 and 30 000 mPas.

2. The stable liquid cosmetic or dermatological cleansing composition as claimed in claim 1, wherein the weight ratio of alkanesulfonate to betaine is from 4:3 to 1:1.

3. The stable liquid cosmetic or dermatological cleansing composition as claimed in claim 1, wherein the viscosity is between 2000 and 10 000 mPas.

4. The stable liquid cosmetic or dermatological cleansing composition of claim 1, wherein the one or more secondary alkanesulfonates and the one or more betaines together an amount is between 6 and 20% by weight, based on the total weight of the composition.

5. The stable liquid cosmetic or dermatological cleansing composition as claimed in claim 4, wherein the amount of the one or more secondary alkanesulfonates and the one or more betaines together is between 8 and 15% by weight, based on the total weight of the composition.

6. The stable liquid cosmetic or dermatological cleansing composition of claim 1, wherein the one or more betaines are selected from the group consisting of alkylamidopropyl-betaines having 8 to 18 carbon atoms in the alkyl group of the fatty acid radical, alkylbetaines having 8 to 18 carbons in the alkyl group and mixtures thereof.

7. The stable liquid cosmetic or dermatological cleansing composition of claim 1, wherein the one or more betaines are selected from alkylamidopropylbetaine or a mixture of alkylamidopropylbetaine and alkylbetaine.

8. The stable liquid cosmetic or dermatological cleansing composition of claim 1, which is clear.

9. The stable liquid cosmetic or dermatological cleansing composition of claim 1, wherein the one or more secondary alkanesulfonates are selected from C₈₋₂₀ alkane-sulfonates.

10. The stable liquid cosmetic or dermatological cleansing composition of claim 1, wherein the one or more secondary alkanesulfonates are selected from C₁₄₋₁₇ alkanesulfonates, or sodium salts thereof.

11. The stable liquid cosmetic or dermatological cleansing composition of claim 1, which contains no ethoxylated or propoxylated compounds.

12. The stable liquid cosmetic or dermatological cleansing composition of claim 1, which contains no alkyl ether sulfates.

13. The stable liquid cosmetic or dermatological cleansing composition of claim 1, which contains one or more cosurfactants.

14. The stable liquid cosmetic or dermatological cleansing composition as claimed in claim 13, wherein the one or more cosurfactants are selected from the group consisting of acylglutamates, alkylpolyglucosides, amphoacetates, coconut monoethanolamides, coconut isopropanolamides, and mixtures thereof.

15. The stable liquid cosmetic or dermatological cleansing composition as claimed in claim 13, wherein the total amount of the one or more cosurfactants, based on the total weight of the composition, is from 6.5 to 20% by weight.

16. The stable liquid cosmetic or dermatological cleansing composition of claim 1, which is a shampoo, shower bath or a liquid soap.
